(19) United States
(12) Patent Application Publication
(10) Pub. No.: US 2026/0187336 A1
(43) Pub. Date: Jul. 2, 2026

(54) METHOD FOR CONVERTING MEDICAL DOCUMENTS, APPARATUS, AND DEVICE

(71) Applicant: Beijing Yilian Zhishu Technology Co., Ltd., Beijing (CN)

(72) Inventor: Shirui WANG, Beijing (CN)

(73) Assignee: Beijing Yilian Zhishu Technology Co., Ltd., Beijing (CN)

(21) Appl. No.: 19/417,569

(22) Filed: Dec. 12, 2025

(30) Foreign Application Priority Data
Dec. 30, 2024 (CN) 202411977767.X

(51) Int. Cl. G06F 40/103; G06F 40/40; G16H 15/00

(52) U.S. Cl. CPC G06F 40/103; G06F 40/40; G16H 15/00

(57) ABSTRACT

Provided is a method for converting medical documents, including: receiving a medical document to be processed input by a user and a target format requirement for the medical document to be processed; determining a target attribute document template based on the target format requirement; inputting the target attribute document template into a large language model to obtain the attribute information output by the large language model; initializing target document conversion parameters for a medical document processing engine based on the attribute information to obtain a target medical document processing engine; and performing format conversion on the medical document to be processed using the target medical document processing engine to obtain a medical document corresponding to the target format requirement. By initializing the target document conversion parameters for the medical document processing engine with the attribute information, the format conversion of medical documents can be achieved.

### CROSS-REFERENCE TO RELATED APPLICATIONS

This application claims priority of Chinese Patent Applicant No. 202411977767.X, documented on Dec. 30, 2024, entitled as “Method for Converting Medical Documents, Apparatus, and Device,” the entire disclosure of which is incorporated herein by reference for all purposes.

### TECHNICAL FIELD

The present disclosure relates to the field of language models, and particularly to a method for converting medical documents, an apparatus, and a device.

### BACKGROUND

With the development of technology, the medical industry is gradually moving towards digitalization, and the systematic organization and efficient application of medical knowledge are becoming key factors in improving the quality of medical services. However, due to the specialized and complex nature of medical knowledge, coupled with the continuous emergence of new research findings and technical standards in the field, the management and updating of medical knowledge has become a challenging task.

In related technologies, medical technicians can organize and classify medical documents so that the medical knowledge within them can be extracted in a standardized form for subsequent tasks. However, different medical documents have varied formats, often requiring a large amount of human and material resources to achieve the extraction of formatted data. Therefore, how to efficiently and conveniently achieve the format conversion of medical documents is a technical problem that technical personnel urgently need to solve.

Embodiments of the present disclosure provide a method for converting medical documents, an apparatus, and a device, which can achieve different format conversions for medical documents.

### SUMMARY

In a first aspect, an embodiment of the present disclosure provides a method for converting medical documents, the method including: receiving a medical document to be processed input by a user and a target format requirement for the medical document to be processed; determining a target attribute document template based on the target format requirement, wherein the target attribute document template includes a preset task prompt for attribute information, and the attribute information corresponds to an attribute of the target format requirement; inputting the target attribute document template into a large language model to obtain the attribute information output by the large language model, wherein the large language model performs a task of generating the attribute information based on the preset task prompt for attribute information; initializing target document conversion parameters for a medical document processing engine based on the attribute information to obtain a target medical document processing engine; and performing format conversion on the medical document to be processed using the target medical document processing engine to obtain a medical document corresponding to the target format requirement.

In a second aspect, an embodiment of the present disclosure provides an apparatus for converting medical documents, the apparatus including:
a receiver, configured to receive a medical document to be processed input by a user and a target format requirement for the medical document to be processed;
a first determiner, configured to determine a target attribute document template based on the target format requirement, wherein the target attribute document template includes a preset task prompt for attribute information, and the attribute information corresponds to an attribute of the target format requirement;
a second determiner, configured to input the target attribute document template into a large language model to obtain the attribute information output by the large language model, wherein the large language model performs a task of generating the attribute information based on the preset task prompt for attribute information;
an initializer, configured to initialize target document conversion parameters for a medical document processing engine based on the attribute information to obtain a target medical document processing engine; and
a converter, configured to perform format conversion on the medical document to be processed using the target medical document processing engine to obtain a medical document corresponding to the target format requirement.

In a third aspect, an embodiment of the present disclosure provides a device for converting medical documents, the device including: a processor and a memory storing computer program instructions; wherein the processor, when executing the computer program instructions, implements the method for converting medical documents according to the first aspect.

In a fourth aspect, an embodiment of the present disclosure provides a non-transitory computer-readable storage medium, on which computer program instructions are stored, wherein the computer program instructions, when executed by a processor, implement the method for converting medical documents according to the first aspect.

In a fifth aspect, an embodiment of the present disclosure provides a computer program product, wherein the instructions in the computer program product, when executed by a processor of an electronic device, cause the electronic device to perform the method for converting medical documents according to the first aspect.

### BRIEF DESCRIPTION OF DRAWINGS

To more clearly explain the technical solutions in the embodiments of the present disclosure, the accompanying drawings required for the embodiments of the present disclosure will be briefly introduced below. For those of ordinary skill in the art, other drawings can be obtained from these drawings without creative effort.

FIG. 1 is a schematic flowchart of a method for converting medical documents according to an embodiment of the present disclosure;

FIG. 2 is a schematic flowchart of a method for converting medical documents according to another embodiment of the present disclosure;

FIG. 3 is a schematic flowchart of a method for converting medical documents according to yet another embodiment of the present disclosure;

FIG. 4 is a schematic flowchart of a method for determining a relevance calculation result according an embodiment of the present disclosure;

FIG. 5 is a schematic flowchart of a method for determining target document content according to an embodiment of the present disclosure;

FIG. 6 is a schematic flowchart of a method for converting medical documents according to yet another embodiment of the present disclosure;

FIG. 7 is a schematic flowchart of a method for converting medical documents according to yet another embodiment of the present disclosure;

FIGS. 8A and B is a schematic flowchart of a method for converting medical documents according to yet another embodiment of the present disclosure;

FIG. 9 is a schematic diagram showing architecture of a system for converting medical documents according to an embodiment of the present disclosure;

FIG. 10 is a schematic structural diagram of a apparatus for converting medical documents according to an embodiment of the present disclosure; and

FIG. 11 is a schematic structural diagram of a device for converting medical documents according to an embodiment of the present disclosure.

### DETAILED DESCRIPTION OF THE EMBODIMENTS

The features and exemplary embodiments of various aspects of the present disclosure will be described in detail below. To make the objectives, technical solutions, and advantages of the present disclosure clearer, the present disclosure will be further described in detail below with reference to the accompanying drawings and specific embodiments. It should be understood that the specific embodiments described herein are only for explaining the present disclosure and not for limiting it. For those skilled in the art, the present disclosure can be implemented without some of these specific details. The following description of the embodiments is merely to provide a better understanding of the present disclosure by showing examples of it.

It should be noted that, relational terms such as “first” and “second” are used merely to distinguish one object or operation from another, and do not necessarily require or imply any such actual relationship or order between these objects or operations. Moreover, the terms “includes,” “including,” or any other variation thereof are intended to cover a non-exclusive inclusion, such that a process, method, article, or device that includes a list of elements does not include only those elements but may include other elements not expressly listed or inherent to such process, method, article, or device. Without more constraints, an element preceded by “includes a . . . ” does not preclude the existence of additional identical elements in the process, method, article, or device that includes the element.

With the digital transformation of the medical industry, the systematic organization and efficient application of medical knowledge have gradually become key factors in improving the quality of medical services. However, due to the professional and complex nature of medical knowledge, coupled with the continuous emergence of new research findings and new technical standards in the field, the management and updating of medical knowledge have become a challenging task.

In related arts, medical technicians can organize and classify medical documents to obtain standardized medical knowledge data. Medical documents can include clinical records from doctors and medical experts, or related medical literature. Medical literature can include books and their photocopies, electronic archives, and other content. It is understandable that different medical personnel have different work habits, so the formats of medical documents vary, which makes format processing very difficult.

Furthermore, different systems may require different formats. Therefore, how to quickly and accurately generate corresponding formats for different systems is also a technical problem that needs to be solved by relevant technical personnel.

To solve the problems in the related arts, embodiments of the present disclosure provide a method for converting medical documents, an apparatus, and a device. The method for converting medical documents provided by the embodiments of the present disclosure will be introduced first.

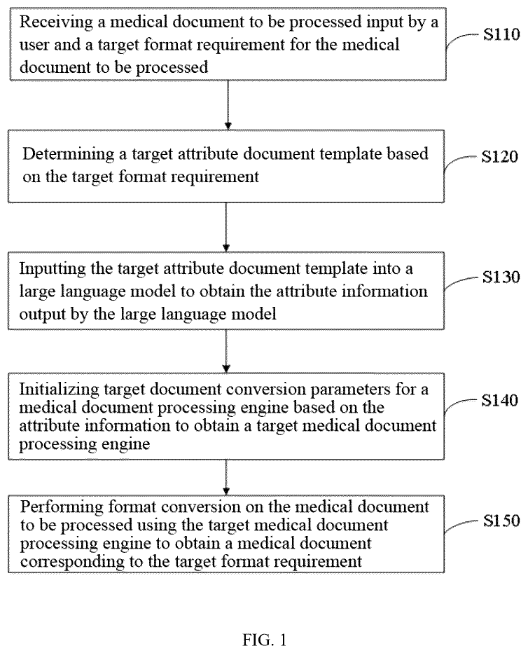

FIG. 1 is a schematic flowchart of a method for converting medical documents according to an embodiment of the present disclosure. As shown in FIG. 1, the method for converting medical documents includes the following steps:

S110, receiving a medical document to be processed input by a user and a target format requirement for the medical document to be processed.

In some embodiments, the medical document to be processed can represent a medical document that needs format conversion. The medical document to be processed can be a medical document entered or imported by a user through a dialog box on the front end of different systems. For example, the medical document to be processed can be a document related to clinical treatment experience for a specific disease; or the medical document to be processed can be a document for case analysis.

In some embodiments, the medical document to be processed input by the user can include one or more documents, and the formats of the medical documents to be processed can be different. In an example, the formats of the medical document to be processed can include online notes, paper notes, diagrams, and temporary drafts. In another example, the medical document to be processed can include documents in multiple languages, such as medical documents in Chinese, English, and other languages.

In some embodiments, the target format requirements can be the format requirements for the format conversion of the document to be processed. In an example, the target format requirements can be selected by the user according to different needs; or, the target format requirements corresponding to the target system can be automatically identified based on the target system selected by the user. The target systems can include patient consultation systems, doctor diagnosis systems, and scientific research query systems.

S120, determining a target attribute document template based on the target format requirement.

The target attribute document template includes a preset task prompt for attribute information, and the attribute information corresponds to the attributes of the target format requirement.

In some embodiments, different target format requirements can correspond to different target attribute document templates. The target attribute document template includes a preset task prompt corresponding to the attribute information.

In an embodiment, in a case analysis task, the target attribute document template can be “illness is [MASK]”. Furthermore, the template can be concatenated with the original text to get the input for the prompt as “I like the Disney films very much. It was [MASK].” It is understandable that, [MASK] can be used to mark the creation of a prediction task. When the model sees [MASK] in the input text, it can predict the word or phrase that should fill the [MASK] position based on the context.

In some embodiments, attribute information can be used to characterize the attribute information to be predicted in the target attribute document template. For example, in a case analysis task, the attribute information can include attributes such as disease type, medical history, and family history.

S130, inputting the target attribute document template into a large language model to obtain the attribute information output by the large language model.

The large language model performs the task of generating attribute information according to the preset task prompt for the attribute information.

In some embodiments, the target attribute document template can be input into the large language model, causing the large language model to perform the task of generating attribute information according to the preset task prompt in the target attribute document template, thereby obtaining the attribute information.

In some embodiments, the large language model (LLM) can be a language model that supports multiple tasks.

S140, initializing target document conversion parameters for a medical document processing engine based on the attribute information to obtain a target medical document processing engine.

In some embodiments, the attribute information determined by the large language model can be used to initialize the target document conversion parameters for the medical document processing engine, thereby obtaining the target medical document processing engine.

In some embodiments, different systems correspond to different medical document processing engines, and the document conversion parameters for the medical document processing engine can be initialized based on different attribute information, enabling the medical document processing engine to process the medical documents to be processed according to different format requirements.

In an embodiment, the medical document processing engine can be a software system or hardware component capable of performing data processing tasks. For example, the medical processing engine can include a search engine, a database processing engine, and a data preprocessing engine, etc.

S150, performing format conversion on the medical document to be processed using the target medical document processing engine to obtain a medical document corresponding to the target format requirement.

In some embodiments, the formatted target medical document processing engine can be used to perform format conversion on the medical document to be processed, thereby obtaining the medical document with the target format requirements.

In the embodiments of this disclosure, by acquiring the medical document to be processed input by the user and the target format requirement corresponding to the medical document, and using the target attribute template corresponding to the target format requirements, the target attribute template is input into a trained large language model, causing the large language model to perform the task of generating attribute information according to the preset task prompt for the attribute information in the target attribute template, thus obtaining the attribute information. Furthermore, the attribute information is used to perform format conversion on the medical document processing engine, thereby obtaining a target medical document processing engine that meets the target format conversion requirement, which is then used to perform format conversion on the medical document to be processed. It can be understood that the method for converting medical documents in the embodiments of the present disclosure can, according to the target format requirement and through a large language model, obtain the initialization parameters corresponding to the medical document processing engine, and initialize the medical document processing engine according to the initialization parameters corresponding to the target format requirements, so that the initialized target medical document processing engine can achieve the format conversion of medical documents. That is, combining with the medical document processing engine to achieve the format conversion of medical documents can quickly, efficiently, and accurately convert the medical document to be processed into the medical document corresponding to the target format requirements.

In some embodiments, different format requirements can correspond to different attribute information, and the attribute information can be used to initialize the parameters of the medical document processing engine. The attribute information can include text attribute information and format attribute information.

To enable the initialization of the medical document processing engine's parameters through attribute information, as another embodiment of the present disclosure, the present disclosure further provides another implementation of the method for converting medical documents.

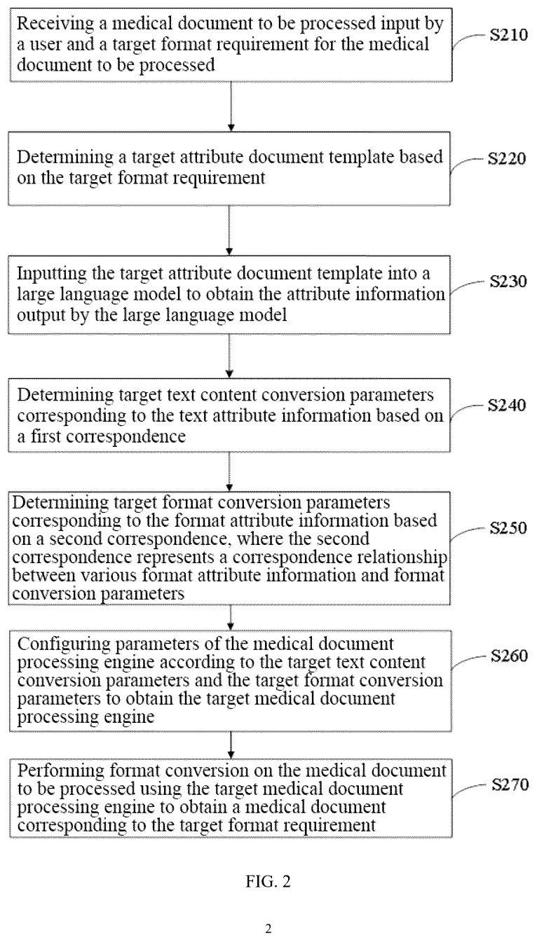

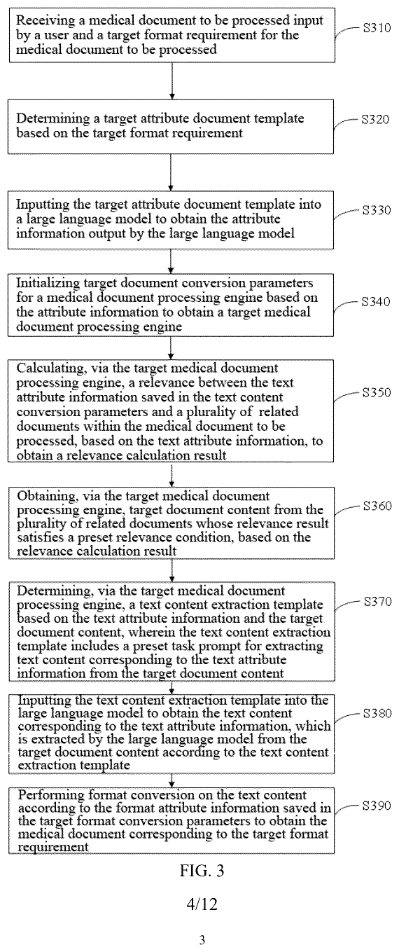

FIG. 2 is a schematic flowchart of a method for converting medical documents according to another embodiment of the present disclosure. As shown in FIG. 2, the method for converting medical documents includes the following steps:

S210, receiving a medical document to be processed input by a user and a target format requirement for the medical document to be processed.

S220, determining a target attribute document template based on the target format requirement.

S230, inputting the target attribute document template into a large language model to obtain the attribute information output by the large language model.

In some embodiments, steps S210-S230 are consistent with steps S110-S130, and step S270 is consistent with step S150, which will not be described in detail here.

S240, determining target text content conversion parameters corresponding to the text attribute information based on a first correspondence.

The first correspondence characterizes the correspondence relationship between various text attribute information and text content conversion parameters.

In some embodiments, the target text content conversion parameters corresponding to the text attribute information in the attribute information can be determined through the first correspondence relationship. The target text content conversion parameters are used to initialize the parameters for the text extraction process in the medical document processing engine.

In an embodiment, the text attribute information can be used to characterize the text features corresponding to the target format requirements. For example, in a case analysis task, the text attribute information can include text features such as disease type, medical history, and family history.

In some embodiments, the first correspondence can be set by relevant technical personnel based on experience or experiments.

S250, determining target format conversion parameters corresponding to the format attribute information based on a second correspondence, where the second correspondence represents a correspondence relationship between various format attribute information and format conversion parameters.

In some embodiments, the target format conversion parameters corresponding to the format attribute information in the attribute information can be determined through the second correspondence. The target format conversion parameters are used to initialize the parameters for the format conversion process in the medical document processing engine.

In an embodiment, the format attribute information can be used to characterize the format requirements corresponding to the target format requirements. Furthermore, the format attribute information can correspond to the text attribute information. For example, in a case analysis task, the format attribute information corresponding to the disease type could be that the disease type is text, the word count is within 256 characters, and the disease type should conform to the enumeration of disease types in the International Classification of Diseases (ICD).

In some embodiments, the second correspondence can be set by relevant technical personnel based on experience or experiments.

S260, configuring parameters of the medical document processing engine according to the target text content conversion parameters and the target format conversion parameters to obtain the target medical document processing engine.

In some embodiments, the parameters of the medical document processing engine can be initialized according to the target text content conversion parameters and the target format conversion parameters determined by the first and second correspondences, to obtain the target medical document processing engine.

S270, performing format conversion on the medical document to be processed using the target medical document processing engine to obtain a medical document corresponding to the target format requirement.

In the embodiments of this disclosure, through the first and second correspondences, the target text content conversion parameters and target format conversion parameters corresponding to the text attribute features and format attribute features of the target format requirements are obtained respectively. These parameters are then used to initialize the medical document processing engine, resulting in a target medical document processing engine that meets the target format requirements. This enables the target medical document processing engine to process the medical document to be processed, ensuring that the document to be processed can be accurately converted by the target medical document processing engine.

To ensure that the medical document to be processed can be accurately converted, as yet another embodiment of the present disclosure, the present disclosure further provides yet another implementation of the method for converting medical documents.

S310, receiving a medical document to be processed input by a user and a target format requirement for the medical document to be processed.

S320, determining a target attribute document template based on the target format requirement.

S330, inputting the target attribute document template into a large language model to obtain the attribute information output by the large language model.

S340, initializing target document conversion parameters for a medical document processing engine based on the attribute information to obtain a target medical document processing engine.

S350, calculating, via the target medical document processing engine, a relevance between the text attribute information saved in the text content conversion parameters and a plurality of related documents within the medical document to be processed, based on the text attribute information, to obtain a relevance calculation result.

S360, obtaining, via the target medical document processing engine, target document content from the plurality of related documents whose relevance result satisfies a preset relevance condition, based on the relevance calculation result.

S370, determining, via the target medical document processing engine, a text content extraction template based on the text attribute information and the target document content, wherein the text content extraction template includes a preset task prompt for extracting text content corresponding to the text attribute information from the target document content.

S380, inputting the text content extraction template into the large language model to obtain the text content corresponding to the text attribute information, which is extracted by the large language model from the target document content according to the text content extraction template.

S390, performing format conversion on the text content according to the format attribute information saved in the target format conversion parameters to obtain the medical document corresponding to the target format requirement.

In some embodiments, steps S310-S340 are consistent with steps S110-S140, which will not be described in detail here.

In some embodiments, in S350, the initialized target medical document processing engine can calculate the relevance between the text attribute information and multiple related documents in the medical document to be processed, based on the text attribute information saved in the text content conversion parameters, to obtain a relevance calculation result.

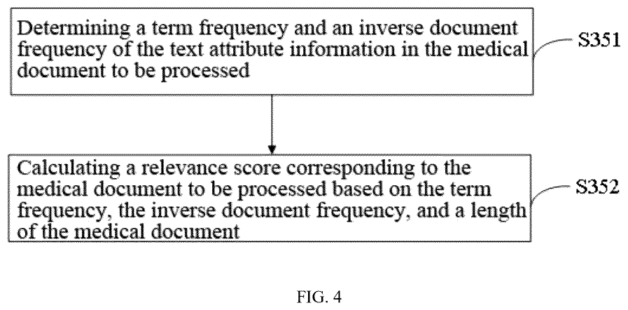

In some embodiments, the related documents can be determined through a relevance calculation formula. FIG. 4 is a schematic flowchart of a method for determining a relevance calculation result according an embodiment of the present disclosure. As shown in FIG. 4, the method for converting medical documents includes the following steps:

S351, determining a term frequency and an inverse document frequency of the text attribute information in the medical document to be processed.

In some embodiments, the term frequency and inverse document frequency (IDF) of the text attribute information in each medical document can be determined.

In an example, the inverse document frequency IDF corresponding to the text attribute information can be calculated using the following formula (1):

$$IDF ( qi ) = log ( \frac{N - n ( qi ) + 0.5}{n ( qi ) + 0.5} ) \qquad ( 1 )$$

Where N is the total number of documents, and n (qi) is the number of documents containing the term “qi”.

S352, calculating a relevance score corresponding to the medical document to be processed based on the term frequency, the inverse document frequency, and a length of the medical document.

In an example, the relevance score for the medical document can be calculated using the following formula (2):

$$score ( D , Q ) = \sum ( IDF ( qi ) \cdot \frac{( k 1 + 1 ) \cdot f ( qi , D )}{k 1 \cdot ( 1 - b + b \cdot \frac{D}{( \text{?} )} ) + f ( qi , d )} ) \qquad ( 2 )$$

$$\text{?} \text{indicates text missing or illegible when filed}$$

Where score (D,Q) represents the relevance score between the medical document D and the query Q, IDF (qi) represents the inverse document frequency of term qi, f (qi,D) represents the frequency of term qi in medical document D, kl and b are adjustable parameters, avgdl represents the average document length, and |D| represents the length of medical document D.

In the embodiments of the present disclosure, by determining the term frequency and inverse document frequency of the text attribute information in the medical document, and calculating the corresponding relevance score for the medical document based on the term frequency, inverse document frequency, and the length of the medical document, the relevance between the target format requirements and each medical document can be determined.

In some embodiments, in S360, the target medical document processing engine can be used to obtain the target document content from multiple related documents where the relevance result satisfies a preset relevance condition, based on the relevance calculation result.

In some embodiments, the target document content can be a medical document or a text segment within a medical document that has a high relevance to the text attribute information. In an example, the medical document with the highest relevance score or a text segment from that medical document can be used as the target document content.

In some embodiments, different target format requirements may correspond to different preset relevance conditions.

In an example, the preset relevance condition can be selecting the most related document from multiple relevant texts as the target document content. For example, the document or text segment with the highest relevance score can be selected as the target document content.

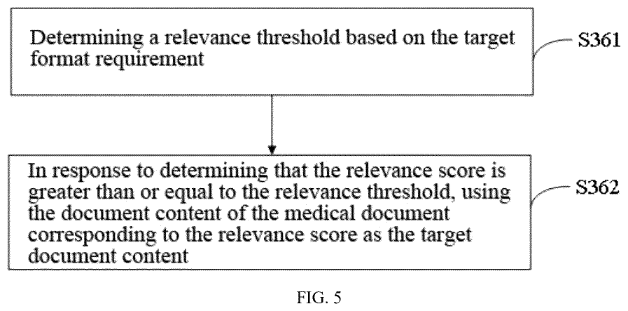

FIG. 5 is a schematic flowchart of a method for determining target document content according to an embodiment of the present disclosure. As shown in FIG. 5, the method for converting medical documents includes the following steps:

S361, determining a relevance threshold based on the target format requirement.

In some embodiments, the relevance threshold can characterize the relevance between the target document content and the text attribute information. In an example, a higher numerical value for the relevance threshold indicates a higher relevance between the target text content and the text attribute information; conversely, a lower numerical value for the relevance threshold indicates a lower relevance between the target text content and the text attribute information.

In some embodiments, different target format requirements correspond to different relevance thresholds. The relevance threshold can be determined by relevant technical personnel based on the different needs of the target format.

S362, in response to determining that the relevance score is greater than or equal to the relevance threshold, using the document content of the medical document corresponding to the relevance score as the target document content.

In some embodiments, when it is determined that the relevance score is greater than or equal to the relevance threshold, the document content of the medical document corresponding to the relevance score can be used as the target document content; when it is determined that the relevance score is less than the relevance threshold, the document content of the medical document corresponding to the relevance score cannot be used as the target document content.

In the embodiments of the present disclosure, by comparing the relevance score with the relevance threshold, it is determined whether the medical document content corresponding to the relevance score is the target document content. The relevance threshold is determined based on different target format requirements, and by comparing the relevance score with the relevance threshold corresponding to different format requirements, the determined target format content can meet different format requirements, thereby improving the accuracy of the format conversion process.

In some embodiments, in S370, the target medical document processing engine can determine a text content extraction template based on the text attribute information and the target document content.

The text content extraction template includes a preset task prompt for extracting text content corresponding to the text attribute information from the target document content.

In some embodiments, the target medical document processing engine can construct a text content extraction target based on the text attribute information and the target document content. The text content extraction template can include preset task prompts, which can be used to extract the text content corresponding to the text attribute information from the target document content.

In an example, when the determined text attribute information is disease type, and the target document content is determined to be related to heart disease, a text content extraction template can be constructed based on the disease type and the heart disease-related content.

In some embodiments, in S380, the text content extraction template is input into a large language model to obtain the text content extracted by the large language model from the target document content, corresponding to the text attribute information, based on the text content extraction template.

In some embodiments, the text content extraction template can be input into a large language model, causing the large language model to predict the corresponding text content based on the text content extraction template. The text content predicted by the large language model corresponds to the text attribute information.

In an example, when the text content extraction template has text attribute information as disease type, the target document content is heart disease-related content, and the text content extraction template is determined, then the predicted text content that the large language model can output could be heart disease.

In some embodiments, in S390, the text content can be format-converted according to the format attribute information saved in the target format conversion parameters to obtain the medical document corresponding to the target format requirements.

In some embodiments, the target medical document processing engine can verify and format-convert the determined text content according to the corresponding format attribute information.

In an example, the predicted text content output by the large language model, such as heart disease, can be verified to determine if it is text content, if it is less than 256 characters, and if it conforms to ICD enumeration examples. After confirming that the content predicted by the large language model meets the requirements, it can be converted according to a preset format, for example, the text content can be converted into text (text, TXT) format. Alternatively, the text content can be vectorized through a pre-trained Chinese text-to-vector model (e.g., text2vec-large-chinese) so that downstream tasks can call the formatted medical document.

In the embodiments of the present disclosure, by obtaining the relevance calculation results of multiple related documents in the medical document to be processed, the target document content is determined, and a text content extraction template is constructed through the target document content and text attribute information. The large language model predicts the text content in the text content extraction template to obtain the corresponding text content. Then, the target medical document processing engine converts the format of the text content according to the format attribute information saved in the target format conversion parameters to obtain the medical document corresponding to the target format requirements. That is, the method of the present disclosure can effectively express and organize the document to be processed, enabling it to achieve correct and standardized format conversion.

To achieve the determination of the attribute template, as another embodiment of the present disclosure, the present disclosure further provides another implementation of the method for converting medical documents.

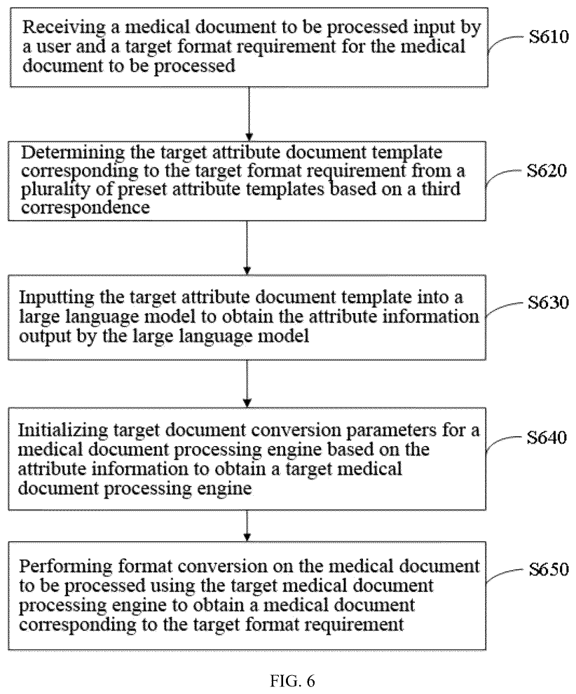

FIG. 6 is a schematic flowchart of a method for converting medical documents according to yet another embodiment of the present disclosure. As shown in FIG. 6, the method for converting medical documents includes the following steps:

S610, receiving a medical document to be processed input by a user and a target format requirement for the medical document to be processed.

In some embodiments, step S610 is consistent with step S110, and steps S630-S650 are consistent with steps S130-S150, which will not be described in detail here.

S620, determining the target attribute document template corresponding to the target format requirement from a plurality of preset attribute templates based on a third correspondence.

The third correspondence represents the correspondence between different preset attribute document templates and format requirements.

In some embodiments, after determining the target format requirements needed by the user, the target attribute document template corresponding to the target format requirements can be determined from multiple preset attribute document templates through the third correspondence.

The preset attribute document templates can be attribute document templates set by technical personnel according to different needs. Furthermore, technical personnel can establish the third correspondence according to the correspondence between the preset attribute document templates and the target format requirements.

In some embodiments, the attribute information corresponding to different preset attribute document templates can be different, or there can be some identical attribute information in different preset attribute document templates. In an example, different preset attribute document templates can include information such as name and gender. In another example, in a template corresponding to a case analysis task, its unique attribute information can include disease type, medical history, and family history.

S630, inputting the target attribute document template into a large language model to obtain the attribute information output by the large language model.

S640, initializing target document conversion parameters for a medical document processing engine based on the attribute information to obtain a target medical document processing engine.

S650, performing format conversion on the medical document to be processed using the target medical document processing engine to obtain a medical document corresponding to the target format requirement.

In the embodiments of the present disclosure, by matching the target format requirements with the format requirements in the third correspondence, the target attribute document template corresponding to the target format requirements is determined, ensuring that the target attribute document template can meet the format requirements needed by different users. It can be understood that in this embodiment of the present disclosure, by determining the target attribute document template corresponding to the target format requirements, the medical document to be processed can be converted into medical documents of various formats, improving the utilization rate of medical documents and providing a good foundation for downstream tasks.

To ensure data transmission security, as another embodiment of the present disclosure, the present disclosure further provides another implementation of the method for converting medical documents.

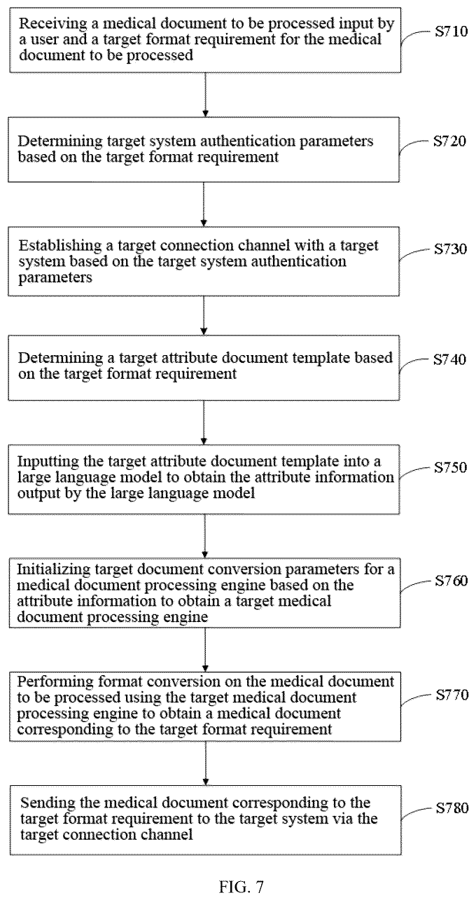

FIG. 7 is a schematic flowchart of a method for converting medical documents according to yet another embodiment of the present disclosure. As shown in FIG. 7, the method for converting medical documents includes the following steps:

S710, receiving a medical document to be processed input by a user and a target format requirement for the medical document to be processed.

In some embodiments, step S710 is consistent with step S110, and steps S740-S770 are consistent with steps S120-S150, which will not be described in detail here.

S720, determining target system authentication parameters based on the target format requirement.

In some embodiments, the target system can be determined based on the target format requirements. The target system can be a system specified by the user. Furthermore, the authentication parameters corresponding to the target system can be determined based on the target system.

In an example, the authentication parameters of the target system's authentication component can be used. For example, the authentication parameters can include credentials for verifying the target system, such as username and password, digital certificates, biometric information, etc.

S730, establishing a target connection channel with a target system based on the target system authentication parameters.

In some embodiments, a target connection channel can be established with the target system when it is determined that the authentication parameters are successfully authenticated. In an example, a connection can be established by configuring connection parameters, such as IP address, port number, encryption protocol, etc., and the security of the communication can be ensured through a handshake process, such as a TLS/SSL handshake.

S740, determining a target attribute document template based on the target format requirement.

S750, inputting the target attribute document template into a large language model to obtain the attribute information output by the large language model.

S760, initializing target document conversion parameters for a medical document processing engine based on the attribute information to obtain a target medical document processing engine.

S770, performing format conversion on the medical document to be processed using the target medical document processing engine to obtain a medical document corresponding to the target format requirement.

S780, sending the medical document corresponding to the target format requirement to the target system via the target connection channel.

In some embodiments, the medical document corresponding to the target format requirements can be sent to the target system using the target connection channel.

In some embodiments, before establishing the connection channel, the system's preset configuration can be invoked to check the system environment and allocate Central Processing Unit (CPU) resources, memory, and storage resources to ensure the operation of the medical document formatting and transmission processes.

In some embodiments, the medical document corresponding to the target format requirements can be compressed using a compression algorithm to increase data transmission speed.

In the embodiments of the present disclosure, a target connection channel can be established with the target system through authentication parameters, thereby enabling the transmission of the medical document corresponding to the target format requirements to the target system. It can be understood that by transmitting data through the target channel, the security of data transmission can be enhanced, ensuring that personal privacy and confidential data are not leaked.

To ensure that the relevant technical structure can be quickly determined, as another embodiment of the present disclosure, the present disclosure further provides another implementation of the method for converting medical documents.

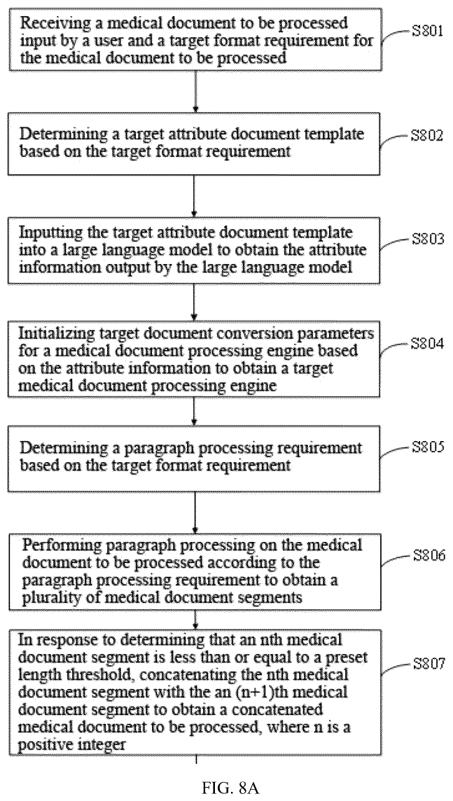

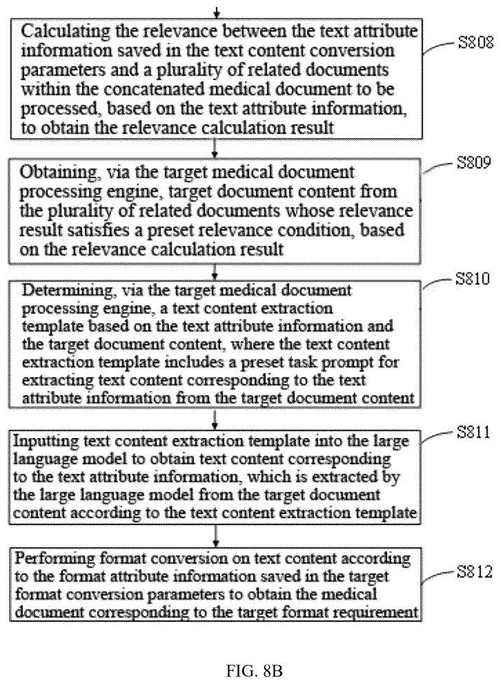

FIGS. 8A and B is a schematic flowchart of a method for converting medical documents according to yet another embodiment of the present disclosure. As shown in FIGS. 8A and B, the method for converting medical documents includes the following steps:

S801, receiving a medical document to be processed input by a user and a target format requirement for the medical document to be processed.

S802, determining a target attribute document template based on the target format requirement.

S803, inputting the target attribute document template into a large language model to obtain the attribute information output by the large language model.

S804, initializing target document conversion parameters for a medical document processing engine based on the attribute information to obtain a target medical document processing engine.

In some embodiments, steps S801-S804 are consistent with steps S310-S340, and steps S809-S812 are consistent with steps S360-S390, so they will not be described in detail here.

S805, determining a paragraph processing requirement based on the target format requirement.

In some embodiments, the paragraph processing requirements are used to characterize the processing requirements for each paragraph in the medical document. Different target format requirements may correspond to different preset paragraph processing requirements.

In some embodiments, the paragraph processing requirements corresponding to the target format requirements can be determined through a preset correspondence. The preset correspondence can characterize the relationship between different format requirements and different paragraph processing requirements.

In an example, the paragraph processing requirements may include paragraph segmentation requirements and requirements for deleting empty lines.

S806, performing paragraph processing on the medical document to be processed according to the paragraph processing requirement to obtain a plurality of medical document segments.

In some embodiments, the medical document to be processed is segmented and empty lines are removed according to the paragraph processing requirements to obtain multiple medical document segments.

S807, in response to determining that an nth medical document segment is less than or equal to a preset length threshold, concatenating the nth medical document segment with the an (n+1)th medical document segment to obtain a concatenated medical document to be processed, where n is a positive integer.

In some embodiments, when it is determined that the nth medical document segment in the medical document segments is less than or equal to a preset length threshold, the nth medical document segment can be concatenated with the (n+1)th medical document segment to obtain a concatenated medical document to be processed. It is understandable that the title segment in a medical document to be processed is often very short, and the following paragraphs are generally used for explanation and detailed description of the title segment. During the retrieval process, it was found that short title segments affect the retrieval results, making it easy to retrieve irrelevant and incomplete short titles. Therefore, merging them with subsequent paragraphs can effectively improve retrieval efficiency and accuracy.

In some embodiments, the preset length threshold can be set by technical personnel based on experiments or relevant experience.

S808, calculating the relevance between the text attribute information saved in the text content conversion parameters and a plurality of related documents within the concatenated medical document to be processed, based on the text attribute information, to obtain the relevance calculation result.

In some embodiments, the relevance between the text attribute information and multiple related documents in the concatenated medical document to be processed can be calculated based on the text attribute information saved in the text content conversion parameters, to obtain a relevance calculation result. It is understandable that performing relevance calculation on the concatenated medical document can effectively improve the efficiency and accuracy of the relevance calculation.

S809, obtaining, via the target medical document processing engine, target document content from the plurality of related documents whose relevance result satisfies a preset relevance condition, based on the relevance calculation result;

S810, determining, via the target medical document processing engine, a text content extraction template based on the text attribute information and the target document content, wherein the text content extraction template includes a preset task prompt for extracting text content corresponding to the text attribute information from the target document content;

S811, inputting the text content extraction template into the large language model to obtain the text content corresponding to the text attribute information, which is extracted by the large language model from the target document content according to the text content extraction template; and

S812, performing format conversion on the text content according to the format attribute information saved in the target format conversion parameters to obtain the medical document corresponding to the target format requirement.

In some embodiments, before concatenating the medical document segments, the medical document to be processed can also undergo processing such as classification and document format normalization. After concatenating the medical document segments, the concatenated medical document can also undergo deduplication and text vectorization processing to facilitate subsequent processes.

In an example, classifying the document to be processed can be based on medical knowledge, storing the documents in different directories. For example, classification categories can be customized by technical personnel, including categories such as basic knowledge of diseases, clinical treatment methods, disease examination plans, and clinical management opinions.

In an example, document format normalization and other processing can handle medical document formats such as compressed packages, text format documents like Word format, Portable Document Format (PDF) documents, txt format documents, and table format documents. Optical Character Recognition (OCR) is used to read the content within the documents and generate medical documents in a unified txt document format.

In an example, deduplicating the information of the medical document to be processed can involve removing redundant information from the information of the medical document to be processed, for example, removing useless information such as the document title at the beginning, table of contents, contacts, contact information, cc, sent, and proofreading.

In an example, performing text vectorization processing on the information of the medical document to be processed can be based on a pre-trained model, vectorizing each text segment in the medical document to be processed and storing it in a vector database (embedding). The vector database contains the document path information (e.g., document classification information) and the vector information of each text segment.

In the embodiments of the present disclosure, by performing corresponding preprocessing on the medical document to be processed, the efficiency and accuracy of relevance calculation are improved, laying a good foundation for subsequent steps.

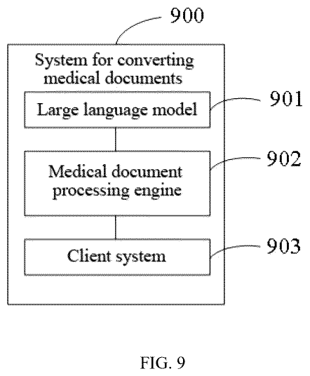

In some embodiments, the method for converting medical documents in the present disclosure is explained in conjunction with FIG. 9 and the following examples.

In some embodiments, the method for converting medical documents can be applied to a medical document conversion system. FIG. 9 is a schematic diagram showing architecture of a system for converting medical documents according to an embodiment of the present disclosure. As shown in FIG. 9, the system for converting medical documents 900 may include a large language model 901, a medical document processing engine 902, and a client system 903.

In some embodiments, a user can enter medical knowledge or import medical documents in the client system 903, i.e., the front-end dialog box. For example: importing “Clinical Management Experience for Special Diseases.doc”. Furthermore, the user can select the target system or target format for the imported medical document or medical knowledge on the front end, where there can be a correspondence between the target system and the target format. Based on the large language model, analyze the requirements of the target format; based on the LLM, construct a template corresponding to the target format requirements through a prompt to obtain the description document of the processing engine (Agent) corresponding to different format requirements, and initialize the processing engine (Agent) according to this description document. The initialized Agent extracts features from the medical knowledge or medical document submitted by the user. Based on the large language model and the requirements of the target format, the content submitted by the user is organized, analyzed, and converted. Through a relevance algorithm, such as the BM25 algorithm, data is extracted and backfilled into the generated target format document. After processing is complete, the system displays the content of the target document for the front-end user to review, or it is automatically reviewed by the system. After the review is completed, the data is output or imported into the target repository through the initialized processing engine.

Based on the method for converting medical documents provided in the above embodiments, the present disclosure further provides a specific implementation of an apparatus for converting medical documents.

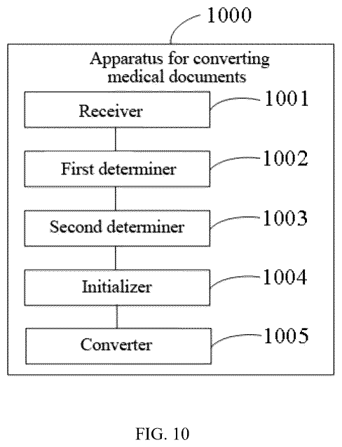

Referring to FIG. 10, the apparatus for converting medical documents 1000 includes the following components:
a receiver 1001, configured to receive a medical document to be processed input by a user and a target format requirement for the medical document to be processed;
a first determiner 1002, configured to determine a target attribute document template based on the target format requirement, wherein the target attribute document template includes a preset task prompt for attribute information, and the attribute information corresponds to an attribute of the target format requirement;
a second determiner 1003, configured to input the target attribute document template into a large language model to obtain the attribute information output by the large language model, wherein the large language model performs a task of generating the attribute information based on the preset task prompt for attribute information;
an initializer 1004, configured to initialize target document conversion parameters for a medical document processing engine based on the attribute information to obtain a target medical document processing engine; and
a converter 1005, configured to perform format conversion on the medical document to be processed using the target medical document processing engine to obtain a medical document corresponding to the target format requirement.

In the embodiments of the present disclosure, by obtaining the medical document to be processed input by the user and its corresponding target format requirements, and using the target attribute template determined by the target format requirements, the target attribute template is input into a trained large language model, causing the large language model to execute the task of generating attribute information according to the preset task prompt for attribute information in the target attribute template, thereby obtaining the attribute information. Furthermore, the attribute information is used to perform format conversion on the medical document processing engine, thus obtaining a target medical document processing engine that meets the target format conversion requirements, and then the target medical document processing engine is used to perform format conversion on the medical document to be processed. It is understandable that the medical document conversion method in this disclosed embodiment can, according to the target format requirements and through a large language model, obtain the initialization parameters corresponding to the medical document processing engine, and initialize the medical document processing engine according to the initialization parameters corresponding to the target format requirements, so that the initialized target medical document processing engine can achieve the format conversion of medical documents.

As an implementation of the present disclosure, the attribute information includes text attribute information and format attribute information, and the initializer 1004 initializes the document conversion parameters of the medical document processing engine based on the attribute information in the following way to obtain the target medical document processing engine: determining target text content conversion parameters corresponding to the text attribute information based on a first correspondence, wherein the first correspondence represents a correspondence relationship between various text attribute information and text content conversion parameters; determining target format conversion parameters corresponding to the format attribute information based on a second correspondence, wherein the second correspondence represents a correspondence relationship between various format attribute information and format conversion parameters; and configuring parameters of the medical document processing engine according to the target text content conversion parameters and the target format conversion parameters to obtain the target medical document processing engine.

As an implementation of the present disclosure, the converter 1005 uses the target medical document processing engine to perform format conversion on the medical document to be processed in the following way to obtain a medical document corresponding to the target format requirements: calculating, via the target medical document processing engine, a relevance between the text attribute information saved in the text content conversion parameters and a plurality of related documents within the medical document to be processed, based on the text attribute information, to obtain a relevance calculation result; obtaining, via the target medical document processing engine, target document content from the plurality of related documents whose relevance result satisfies a preset relevance condition, based on the relevance calculation result; determining, via the target medical document processing engine, a text content extraction template based on the text attribute information and the target document content, wherein the text content extraction template includes a preset task prompt for extracting text content corresponding to the text attribute information from the target document content; inputting the text content extraction template into the large language model to obtain the text content corresponding to the text attribute information, which is extracted by the large language model from the target document content according to the text content extraction template; and performing format conversion on the text content according to the format attribute information saved in the target format conversion parameters to obtain the medical document corresponding to the target format requirement.

As an implementation of the present disclosure, the converter 1005 calculates the relevance between the text attribute information and multiple related documents in the medical document to be processed based on the text attribute information saved in the text content conversion parameters in the following way to obtain a relevance calculation result: determining a term frequency and an inverse document frequency of the text attribute information in the medical document to be processed; and calculating a relevance score corresponding to the medical document to be processed based on the term frequency, the inverse document frequency, and a length of the medical document.

As an implementation of the present disclosure, the converter 1005 obtains the target document content whose relevance result satisfies a preset relevance condition from multiple related documents through the target medical document processing engine based on the relevance calculation result in the following way: determining a relevance threshold based on the target format requirement; and in response to determining that the relevance score is greater than or equal to the relevance threshold, using the document content of the medical document corresponding to the relevance score as the target document content.

As an implementation of the present disclosure, the first determiner 1002 determines the attribute document template based on the target format requirements in the following way: determining the target attribute document template corresponding to the target format requirement from a plurality of preset attribute templates based on a third correspondence, wherein the third correspondence represents a correspondence relationship between various preset attribute templates and format requirements.

In an embodiment, the apparatus further includes: an authenticator, configured to determine target system authentication parameters based on the target format requirements; establish a target connection channel with the target system based on the target system authentication parameters. The apparatus further includes a sender, configured to, after using the target medical document processing engine to perform format conversion on the medical document to be processed to obtain the medical document corresponding to the target format requirements, send the medical document corresponding to the target format requirements to the target system through the target connection channel.

In an embodiment, before calculating the correlation between the text attribute information and multiple related documents in the medical document to be processed based on the text attribute information saved in the text content conversion parameters to obtain the correlation calculation result, the initializer 1004 is further configured to: determine a paragraph processing requirement based on the target format requirements; perform paragraph processing on the medical document to be processed according to the paragraph processing requirement to obtain multiple medical document segments; and when it is determined that the nth medical document segment is less than or equal to a preset length threshold, concatenate the nth medical document segment with the (n+1)th medical document segment to obtain a concatenated medical document to be processed, where n is a positive integer; the calculating the correlation between the text attribute information and multiple related documents in the medical document to be processed based on the text attribute information saved in the text content conversion parameters to obtain the correlation calculation result includes: calculating the correlation between the text attribute information and multiple related documents in the concatenated medical document to be processed based on the text attribute information saved in the text content conversion parameters to obtain the correlation calculation result.

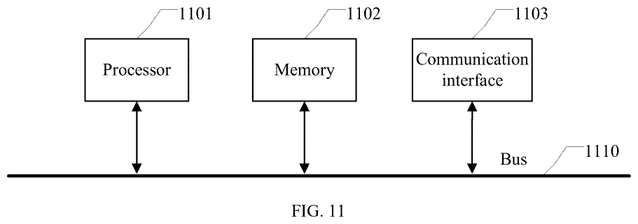

FIG. 11 is a schematic structural diagram of a device for converting medical documents according to an embodiment of the present disclosure.

As shown in FIG. 11, the device for converting medical documents can include a processor 1101 and a memory 1102 storing computer program instructions.

In an embodiment, the aforementioned processor 1101 can include a central processing unit (CPU), or an Application Specific Integrated Circuit (ASIC), or can be configured as one or more integrated circuits for implementing the embodiments of the present disclosure.

The memory 1102 can include mass storage for data or instructions. By way of example and not limitation, the memory 1102 may include a Hard Disk Drive (HDD), a floppy disk drive, flash memory, an optical disc, a magneto-optical disc, a magnetic tape, or a Universal Serial Bus (USB) drive, or a combination of two or more of these. Where appropriate, the memory 1102 may include removable or non-removable (or fixed) media. Where appropriate, the memory 1102 may be internal or external to the integrated gateway disaster recovery device. In a particular embodiment, the memory 1102 is a non-volatile solid-state memory.

In an embodiment, the memory may include a read-only memory (ROM), random access memory (RAM), disk storage media devices, optical storage media devices, flash memory devices, electrical, optical, or other physical/tangible memory storage devices. Therefore, generally, the memory includes one or more tangible (non-transitory) computer-readable storage media (e.g., a memory device) encoded with software including computer-executable instructions, and when the software is executed (e.g., by one or more processors), it is operable to perform the operations described with reference to the methods according to the first aspect of the present disclosure.

The processor 1101 implements any of the methods for converting medical documents in the above embodiments by reading and executing the computer program instructions stored in the memory 1102.

In an embodiment, the device for converting medical documents may also include a communication interface 1103 and a bus 1110. As shown in FIG. 11, the processor 1101, the memory 1102, and the communication interface 1103 are connected and communicate with each other via the bus 1110.

The communication interface 1103 is mainly used to implement communication between the various modules, apparatuses, units, and/or devices in the embodiments of the present disclosure.

The bus 1110 includes hardware, software, or both, coupling the components of the device for converting medical documents to each other. By way of example and not limitation, the bus may include an Accelerated Graphics Port (AGP) or other graphics bus, an Enhanced Industry Standard Architecture (EISA) bus, a Front-Side Bus (FSB), a HyperTransport (HT) interconnect, an Industry Standard Architecture (ISA) bus, an InfiniBand interconnect, a Low Pin Count (LPC) bus, a memory bus, a Micro Channel Architecture (MCA) bus, a Peripheral Component Interconnect (PCI) bus, a PCI-Express (PCI-X) bus, a Serial Advanced Technology Attachment (SATA) bus, a Video Electronics Standards Association Local (VLB) bus, or other suitable bus, or a combination of two or more of the above. Where appropriate, the bus 1110 may include one or more buses. Although the embodiments of the present disclosure describe and illustrate specific buses, the present disclosure may contemplate any suitable bus or interconnect.

Additionally, in conjunction with the method for converting medical documents in the above embodiments, the embodiments of the present disclosure may provide a non-transitory computer storage medium to implement them. The computer storage medium stores computer program instructions; when the computer program instructions are executed by a processor, they implement any of the methods in the above embodiments.

An embodiment of the present disclosure further provides a computer program product, which includes a computer program. When the computer program is executed by a processor, it implements any methods for converting medical documents in the above embodiments.

It should be clarified that the present disclosure is not limited to the specific configurations and processes described above and shown in the drawings. For the sake of brevity, detailed descriptions of known methods are omitted here. In the above embodiments, several specific steps are described and shown as examples. However, the method process of the present disclosure is not limited to the specific steps described and shown. Those skilled in the art can make various changes, modifications, and additions, or change the order between steps, after understanding the principle of the present disclosure.

The functional blocks shown in the structural block diagrams described above may be implemented as hardware, software, firmware, or a combination thereof. When implemented in hardware, it can be, for example, an electronic circuit, an application specific integrated circuit (ASIC), appropriate firmware, a plug-in, a function card, and so on. When implemented in software, the elements of the present disclosure are programs or code segments used to perform the required tasks. The program or code segments can be stored in a machine-readable medium or transmitted over a transmission medium or communication link via a data signal carried in a carrier wave. A “machine-readable medium” may include any medium capable of storing or transmitting information. Examples of machine-readable media include electronic circuits, semiconductor memory devices, read-only memory (ROM), flash memory, erasable read only memory (EROM), floppy disks, compact disc read-only memory (CD-ROM), optical disks, hard disks, fiber optic media, radio frequency (RF) links, and so on. The code segments may be downloaded via computer networks such as the Internet, intranets, etc.

It also needs to be stated that the exemplary embodiments mentioned in the present disclosure describe some methods or systems based on a series of steps or devices. However, the present disclosure is not limited to the order of the steps described above, that is, the steps can be executed in the order mentioned in the embodiments, or in a different order from the embodiments, or several steps can be executed simultaneously.

The various aspects of the present disclosure have been described above with reference to flowcharts and/or block diagrams of methods, apparatuses (systems), and computer program products according to embodiments of the present disclosure. It should be understood that each block in the flowcharts and/or block diagrams, and combinations of blocks in the flowcharts and/or block diagrams, can be implemented by computer program instructions. These computer program instructions may be provided to the processor of a general-purpose computer, a special-purpose computer, or other programmable data processing apparatus to produce a machine, such that these instructions, when executed via the processor of the computer or other programmable data processing apparatus, enable the implementation of the functions/actions specified in one or more blocks of the flowchart and/or block diagram. Such a processor may be, but is not limited to, a general-purpose processor, a special-purpose processor, a special application processor, or a field-programmable logic circuit. It can also be understood that each block in the block diagrams and/or flowcharts, and combinations of blocks in the block diagrams and/or flowcharts, can also be implemented by dedicated hardware that performs the specified functions or actions, or can be implemented by a combination of dedicated hardware and computer instructions.

The foregoing are only specific embodiments of the present disclosure. It can be clearly understood by those skilled in the art that, for the convenience and brevity of description, the specific working processes of the systems, modules, and units described above can be referred to the corresponding processes in the foregoing method embodiments, and will not be repeated here. It should be understood that the scope of protection of the present disclosure is not limited to this. Any person skilled in the art can easily conceive of various equivalent modifications or replacements within the technical scope disclosed by the present disclosure, and these modifications or replacements should be covered within the scope of protection of the present disclosure.

What is claimed is:

1. A method for converting medical documents, comprising:
receiving a medical document to be processed input by a user and a target format requirement for the medical document to be processed;
determining a target attribute document template based on the target format requirement, wherein the target attribute document template comprises a preset task prompt for attribute information, and the attribute information corresponds to an attribute of the target format requirement;
inputting the target attribute document template into a large language model to obtain the attribute information output by the large language model, wherein the large language model performs a task of generating the attribute information based on the preset task prompt for attribute information;
initializing target document conversion parameters for a medical document processing engine based on the attribute information to obtain a target medical document processing engine; and
performing format conversion on the medical document to be processed using the target medical document processing engine to obtain a medical document corresponding to the target format requirement.

2. The method according to claim 1, wherein the attribute information comprises text attribute information and format attribute information; and
the initializing target document conversion parameters for a medical document processing engine based on the attribute information to obtain a target medical document processing engine comprises:
determining target text content conversion parameters corresponding to the text attribute information based on a first correspondence, wherein the first correspondence represents a correspondence relationship between various text attribute information and text content conversion parameters;
determining target format conversion parameters corresponding to the format attribute information based on a second correspondence, wherein the second correspondence represents a correspondence relationship between various format attribute information and format conversion parameters; and
configuring parameters of the medical document processing engine according to the target text content conversion parameters and the target format conversion parameters to obtain the target medical document processing engine.

3. The method according to claim 1, wherein the performing format conversion on the medical document to be processed using the target medical document processing engine to obtain a medical document corresponding to the target format requirement comprises:
calculating, via the target medical document processing engine, a relevance between the text attribute information saved in the text content conversion parameters and a plurality of related documents within the medical document to be processed, based on the text attribute information, to obtain a relevance calculation result;
obtaining, via the target medical document processing engine, target document content from the plurality of related documents whose relevance result satisfies a preset relevance condition, based on the relevance calculation result;
determining, via the target medical document processing engine, a text content extraction template based on the text attribute information and the target document content, wherein the text content extraction template comprises a preset task prompt for extracting text content corresponding to the text attribute information from the target document content;
inputting the text content extraction template into the large language model to obtain the text content corresponding to the text attribute information, which is extracted by the large language model from the target document content according to the text content extraction template; and
performing format conversion on the text content according to the format attribute information saved in the target format conversion parameters to obtain the medical document corresponding to the target format requirement.

4. The method according to claim 3, wherein the calculating a relevance between the text attribute information saved in the text content conversion parameters and a plurality of related documents within the medical document to be processed, based on the text attribute information, to obtain a relevance calculation result comprises:
determining a term frequency and an inverse document frequency of the text attribute information in the medical document to be processed; and
calculating a relevance score corresponding to the medical document to be processed based on the term frequency, the inverse document frequency, and a length of the medical document.

5. The method according to claim 4, wherein the obtaining, via the target medical document processing engine, target document content from the plurality of related documents whose relevance result satisfies a preset relevance condition, based on the relevance calculation result comprises:
determining a relevance threshold based on the target format requirement; and
in response to determining that the relevance score is greater than or equal to the relevance threshold, using the document content of the medical document corresponding to the relevance score as the target document content.

6. The method according to claim 1, wherein the determining a attribute document template based on the target format requirement comprises:
determining the target attribute document template corresponding to the target format requirement from a plurality of preset attribute templates based on a third correspondence, wherein the third correspondence represents a correspondence relationship between various preset attribute templates and format requirements.

7. The method according to claim 1, further comprising:
determining target system authentication parameters based on the target format requirement; and
establishing a target connection channel with a target system based on the target system authentication parameters;
wherein after performing format conversion on the medical document to be processed using the target medical document processing engine to obtain a medical document corresponding to the target format requirement, the method further comprises:
sending the medical document corresponding to the target format requirement to the target system via the target connection channel.

8. The method according to claim 3, wherein before the calculating, via the target medical document processing engine, a relevance between the text attribute information saved in the text content conversion parameters and a plurality of related documents within the medical document to be processed, based on the text attribute information, to obtain a relevance calculation result, the method further comprises:
determining a paragraph processing requirement based on the target format requirement;
performing paragraph processing on the medical document to be processed according to the paragraph processing requirement to obtain a plurality of medical document segments; and
in response to determining that an nth medical document segment is less than or equal to a preset length threshold, concatenating the nth medical document segment with the an (n+1)th medical document segment to obtain a concatenated medical document to be processed, where n is a positive integer;
and wherein the calculating, via the target medical document processing engine, a relevance between the text attribute information saved in the text content conversion parameters and a plurality of related documents within the medical document to be processed, based on the text attribute information, to obtain a relevance calculation result comprises:
calculating the relevance between the text attribute information saved in the text content conversion parameters and a plurality of related documents within the concatenated medical document to be processed, based on the text attribute information, to obtain the relevance calculation result.

9. An apparatus for converting medical documents, the apparatus comprising:
a receiver, configured to receive a medical document to be processed input by a user and a target format requirement for the medical document to be processed;
a first determiner, configured to determine a target attribute document template based on the target format requirement, wherein the target attribute document template comprises a preset task prompt for attribute information, and the attribute information corresponds to an attribute of the target format requirement;
a second determiner, configured to input the target attribute document template into a large language model to obtain the attribute information output by the large language model, wherein the large language model performs a task of generating the attribute information based on the preset task prompt for attribute information;
an initializer, configured to initialize target document conversion parameters for a medical document processing engine based on the attribute information to obtain a target medical document processing engine; and
a converter, configured to perform format conversion on the medical document to be processed using the target medical document processing engine to obtain a medical document corresponding to the target format requirement.

10. A device for converting medical documents, comprising a processor, and a memory storing computer program instructions; wherein the processor, when executing the computer program instructions, performs acts comprising:
receiving a medical document to be processed input by a user and a target format requirement for the medical document to be processed;
determining a target attribute document template based on the target format requirement, wherein the target attribute document template comprises a preset task prompt for attribute information, and the attribute information corresponds to an attribute of the target format requirement;
inputting the target attribute document template into a large language model to obtain the attribute information output by the large language model, wherein the large language model performs a task of generating the attribute information based on the preset task prompt for attribute information;
initializing target document conversion parameters for a medical document processing engine based on the attribute information to obtain a target medical document processing engine; and
performing format conversion on the medical document to be processed using the target medical document processing engine to obtain a medical document corresponding to the target format requirement.
